SEQUENCE LISTING (8 sequences)

SEQ ID NO: 1 - protein, 214 aa, Artificial Sequence [sequence omitted]

SEQ ID NO: 2 - protein, 447 aa, Artificial Sequence
  heavy chain variable region
VQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 11 aa, Artificial Sequence
  LCDR1
RASQDISSYLN

SEQ ID NO: 4 - protein, 7 aa, Artificial Sequence
  LCDR2
YTSRLHS

SEQ ID NO: 5 - protein, 9 aa, Artificial Sequence
  LCDR3
QQGNTLPYT

SEQ ID NO: 6 - protein, 6 aa, Artificial Sequence
  HCDR1
SDHAWS

SEQ ID NO: 7 - protein, 15 aa, Artificial Sequence
  HCDR2
YISYSGITTYNPSLK

SEQ ID NO: 8 - protein, 10 aa, Artificial Sequence
  HCDR3
SLARTTAMDY

(19) United States
(12) Patent Application Publication
(10) Pub. No.: US 2022/0242959 A1
(43) Pub. Date: Aug. 4, 2022

(54) MODIFIED DOSAGE OF SUBCUTANEOUS TOCILIZUMAB FOR RHEUMATOID ARTHRITIS

(71) Applicants: SANOFI BIOTECHNOLOGY, Paris (FR); REGENERON PHARMACEUTICALS, INC., Tarrytown, NY (US)

(72) Inventors: Chieh-I CHEN, Tarrytown, NY (US); Wenhui WEI, Tarrytown, NY (US)

(21) Appl. No.: 17/615,732

(22) PCT Filed: Jun. 11, 2020
(86) PCT No.: PCT/US2020/037325

§ 371 Date: Dec. 1, 2021

Related U.S. Application Data
(60) Provisional application No. 62/860,611, filed on Jun. 12, 2019.

(30) Foreign Application Priority Data
Feb. 27, 2020 (EP) 20305192.5

(51) Int. Cl. C07K 16/28; A61P 19/02; A61P 37/06; A61K 31/519; A61K 39/395; C07K 16/24

(52) U.S. Cl. CPC C07K 16/2866; A61P 19/02; A61P 37/06; A61K 2039/545; A61K 39/3955; C07K 16/241; A61K 31/519

(57) ABSTRACT

The present disclosure relates the dosage modification and choice of an IL6 antibody for the treatment of rheumatoid arthritis in subjects.

### RELATED APPLICATIONS

This application claims the benefit of U.S. Provisional Application No. 62/860,611 filed on Jun. 12, 2019, and E.P. Application 20305192.5 filed on Feb. 27, 2020, the entire disclosures of which are hereby incorporated herein by reference.

### SEQUENCE LISTING

The instant application contains a Sequence Listing which has been submitted electronically in ASCII format and is hereby incorporated by reference in its entirety. Said ASCII copy, created on Jun. 11, 2020, is named 706369_Sequence_Listing.txt and is 11 kilobytes in size.

### BACKGROUND

Management of rheumatoid arthritis (RA) is primarily based on the use of disease modifying antirheumatic drugs (DMARDs). Current guidelines recommend conventional synthetic DMARDs (csDMARDs) as first-line treatment, with the aim of achieving disease remission or reducing disease activity. Although csDMARDs form the basis of care in RA, a proportion of patients with moderate-to-severe RA fail to respond to csDMARDs. In such cases, the guidelines recommend initiating a biologic DMARD (bDMARD) in combination with a csDMARD.

Multiple bDMARDs are available for the treatment of RA. Tocilizumab (TCZ) is a humanized anti-interleukin-6 (IL-6) receptor monoclonal antibody that binds to the membrane-bound and soluble IL-6 receptors, inhibiting IL-6 signaling. TCZ is indicated for monotherapy or in combination with csDMARDs for the treatment of patients with moderate-to-severe active RA who have had an inadequate response to >1 DMARDs.

### SUMMARY

Various aspects provided herein present a method of treating Rheumatoid arthritis (RA) using an antibody that specifically binds to the IL-6 receptor comprises a heavy chain variable region sequence of SEQ ID NO: 2 and a light chain variable region sequence of SEQ ID NO: 1.

In various embodiments, the antibody comprises a heavy chain variable region (VH) and a light chain variable region (VL), wherein the VH comprises the three complementarity determining regions (CDRs) found within the sequence of SEQ ID NO:1 and wherein the VL comprises the three CDRs found within the sequence of SEQ ID NO:2. In various embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO: 6; the HCDR2 comprises the amino acid sequence of SEQ ID NO: 7; the HCDR3 comprises the amino acid sequence of SEQ ID NO: 8; the LCDR1 comprises the amino acid sequence of SEQ ID NO: 3; the LCDR2 comprises the amino acid sequence of SEQ ID NO: 4; and the LCDR3 comprises the amino acid sequence of SEQ ID NO: 5.

In various embodiments, the antibody is tocilizumab.

In various embodiments, this disclosure presents method of administering to a subject in need thereof, an IL-6 receptor antibody as described above (e.g., tocilizumab), comprising selecting a subject who has not previously been administered the IL-6 receptor antibody, or who has been administered the IL-6 receptor antibody for less than three months, and who does not have anemia; and administering 162 mg of the IL-6 receptor antibody, once per week to the subject, wherein the IL-6 receptor antibody is administered subcutaneously; or 8 mg/kg of the IL-6 receptor antibody, once every 4 weeks to the subject, wherein the IL-6 receptor antibody is administered intravenously.

In various embodiments, this disclosure presents a method of treating rheumatoid arthritis in a subject in need thereof, comprising selecting a subject who has not previously been administered the IL-6 receptor antibody, or who has been administered the IL-6 receptor antibody for less than three months, and who is from 18 to 34 years old; and administering (a) 162 mg of the IL-6 receptor antibody, once per week to the subject, wherein the IL-6 receptor antibody, is administered subcutaneously; or (b) 8 mg/kg of the IL-6 receptor antibody, once every 4 weeks to the subject, wherein the IL-6 receptor antibody, is administered intravenously.

In various embodiments, this disclosure presents a method of treating rheumatoid arthritis in a subject in need thereof, comprising selecting a subject who has not previously been administered the IL-6 receptor antibody, or who has been administered the IL-6 receptor antibody for less than three months, and who has not been administered a corticosteroid within 90 days; and administering (a) 162 mg of the IL-6 receptor antibody, once per week to the subject, wherein the antibody is administered subcutaneously; or (b) 8 mg/kg of the IL-6 receptor antibody, once every 4 weeks to the subject, wherein the antibody is administered intravenously.

In various embodiments, this disclosure presents a method of treating rheumatoid arthritis in a subject in need thereof, comprising selecting a subject who has not previously been administered the IL-6 receptor antibody, or who has been administered the IL-6 receptor antibody for less than three months, and who has depression; and administering (a) 162 mg of the IL-6 receptor antibody, once per week to the subject, wherein the IL-6 receptor antibody is administered subcutaneously; or (b) 8 mg/kg of the IL-6 receptor antibody, once every 4 weeks to the subject, wherein the IL-6 receptor antibody is administered intravenously.

In various embodiments, the method comprises administering 162 mg of the IL-6 receptor antibody, once per week to the subject, subcutaneously. In various embodiments, the method comprises administering 8 mg/kg of the IL-6 receptor antibody once every 4 weeks to the subject, intravenously.

In various embodiments, the subject has moderately-to-severely active rheumatoid arthritis. In various embodiments, the subject has not been administered sarilumab. In various embodiments, the subject weighs less than 100 kg. In various embodiments, the subject does not have ankylosing spondylitis, Crohn's disease, juvenile idiopathic arthritis, psoriasis, psoriatic arthritis, ulcerative colitis, chronic lymphocytic leukemia, non-Hodgkin's lymphoma, or giant-cell arteritis.

In some embodiments, the subject is selected if the subject does not have anemia and is from 18 to 34 years old. In some embodiments, the subject is selected if the subject does not have anemia and has not been administered a corticosteroid within 90 days. In various embodiments, the subject is selected if the subject is from 18 to 34 years old and has not been administered a corticosteroid within 90 days. In various embodiments, the subject is selected if the subject does not have anemia, has not been administered a corticosteroid within 90 days, and is from 18 to 34 years old. In various embodiments, subject is selected if the subject does not have anemia and has depression.

In various embodiments, the subject is selected if the subject has depression and has not been administered a corticosteroid within 90 days. In some embodiments, the subject is selected if the subject is from 18 to 34 years old and has depression. In some embodiments, the subject is selected if the subject does not have anemia, has depression and is from 18 to 34 years old. In various embodiments, the subject is selected if the subject does not have anemia, has depression, and has not been administered a corticosteroid within 90 days. In various embodiments, the subject is selected if the subject is from 18 to 34 years old, has depression, and has not been administered a corticosteroid within 90 days. In various embodiments the subject is selected if the subject does not have anemia, has not been administered a corticosteroid within 90 days, is from 18 to 34 years old, and has depression.

In various embodiments the subject is within 90 days is within 90 days of the subject's first administration of the IL-6 receptor antibody. In various embodiments, the subject is within 90 days is within 90 days of the selection. In various embodiments, the corticosteroid is prednisone.

In various embodiments, the subject is not administered any other DMARD in course of administration with the IL-6 receptor antibody. In various embodiments, wherein the subject is administered one or more additional DMARDs with the IL-6 receptor antibody. In various embodiments, the one or more additional DMARDs comprise methotrexate. In various embodiments, the subject previously had an inadequate response to a conventional synthetic DMARD or a biologic DMARD. In various embodiments, wherein the conventional synthetic DMARD is methotrexate. In some embodiments, the biologic DMARD is a TNFα inhibitor. In various embodiments, the TNFα inhibitor is adalimumab.

In various embodiments, the subject has not previously been administered the IL-6 receptor antibody. In various embodiments, the subject has been administered the IL-6 receptor antibody for less than three months. In various embodiments, the subject has been administered the IL-6 receptor antibody for less than two months. In various embodiments, the subject has been administered the IL-6 receptor antibody for less than one month.

In various embodiments, the subject is a female.

In various embodiments, the IL-6 receptor antibody is tocilizumab

### BRIEF DESCRIPTION OF FIGURES

FIG. 1 shows Attrition Flow Chart for Truven MarketScan and Optum Clinformatics Patients.

FIG. 2A shows a Kaplan-Meier analysis for time to first dose escalation for SC TCZ in Truven patients. FIG. 2B shows the same analysis for Optum patients.

### DETAILED DESCRIPTION

TCZ can be administered subcutaneously (SC) or as an intravenous (IV) infusion. The United States prescribing information recommends different dosing regimens depending on whether a patient receives IV or SC injection of TCZ. The recommended dosing regimen for IV administration is 4 mg/kg every 4 weeks, followed by an increase to 8 mg/kg every 4 weeks based on clinical response. The recommended dosing regimen for SC administration differs depending on the patient's weight. In patients weighing <100 kg, TCZ is administered at 162 mg every 2 weeks (Q2W), while in patients weighing >100 kg, TCZ, is administered at 162 mg every week (QW). Based on the patient's clinical response, and at the physician's discretion, patients starting on the lower dose of 162 mg Q2W may be up-titrated to SC TCZ 162 mg QW, and US guidelines recommend that therapeutic agents should be given for at least 3 months before therapy escalation is considered.

Although physicians can tailor the dosage of IV and SC TCZ based on clinical response, real-world data demonstrating actual dose modifications among patients receiving SC TCZ are scarce.

The present disclosure provides data showing that certain subject populations are more likely to require dose escalation when receiving treatment with tocilizumab (TCZ). In various embodiments, these subject populations from rheumatoid arthritis (RA). In various embodiments, RA subjects who are female, do not have anemia, are from 18 to 34 years old, have not been administered a corticosteroid within 90 days and/or have depression start treatment at the higher dose of TCZ, rather than receiving a lower dose that is then escalated. In various embodiments, RA subjects who are female, do not have anemia, are from 18 to 34 years old, have not been administered a corticosteroid within 90 days and/or have depression are treated by administering the escalated dose of TCZ within 3 months of beginning of therapy with TCZ.

In various embodiments, a non-escalated dose of TCZ is less than 8 mg/kg administered intravenously (IV) once every four weeks. In various embodiments, a non-escalated dose of TCZ is less than 162 mg administered subcutaneously (SC) once every two weeks. In various embodiments, a non-escalated dose of TCZ is 4 mg/kg administered IV once every four weeks. In various embodiments, a non-escalated dose of TCZ is than 162 mg administered SC once every two weeks.

In various embodiments, an escalated dose of TCZ is at least 8 mg/kg administered intravenously (IV) once every four weeks. In various embodiments, an escalated dose of TCZ is at least 162 mg administered SC once every week. In various embodiments, an escalated dose of TCZ is 8 mg/kg administered IV once every four weeks. In various embodiments, an escalated dose of TCZ is 162 mg administered SC once every week.

As used within the claims, the Summary, and the Detailed Description herein, the term “about” in quantitative terms refers to plus or minus 10% of the value it modifies (rounded up to the nearest whole number if the value is not sub-dividable, such as a number of molecules or nucleotides). For example, the phrase “about 100 mg” would encompass 90 mg to 110 mg, inclusive; the phrase “about 2500 mg” would encompass 2250 mg to 2750 mg. When applied to a percentage, the term “about” refers to plus or minus 10% relative to that percentage. For example, the phrase “about 20%” would encompass 18-22% and “about 80%” would encompass 72-88%, inclusive. Moreover, where “about” is used herein in conjunction with a quantitative term it is understood that in addition to the value plus or minus 10%, the exact value of the quantitative term is also contemplated and described. For example, the term “about 23%” expressly contemplates, describes, and includes exactly 23%.

It is to be noted that the term “a” or “an” entity refers to one or more of that entity; for example, “a symptom,” is understood to represent one or more symptoms. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

### Antibodies

The present disclosure includes methods that comprise administering to a subject an antibody, or an antigen-binding fragment thereof, that binds specifically to hIL-6R. As used herein, the term “hIL-6R” means a human cytokine receptor that specifically binds human interleukin-6 (IL-6). In certain embodiments, the antibody that is administered to the patient binds specifically to the extracellular domain of hIL-6R.

The term “antibody”, as used herein, refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, and bivalent nanobodies), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may in various embodiments consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may in various embodiments comprise a homo-dimer or hetero-dimer (or other multimer) of any of the variable and constant domain configurations listed above in non-covalent association with one another and/or with one or more monomeric VH or VL domain (e.g., by disulfide bond(s)).

In certain embodiments, the antibody or antibody fragment for use in a method disclosed herein may be a monospecific antibody. In certain embodiments, the antibody or antibody fragment for use in a method disclosed herein may be a multispecific antibody, which may be specific for different epitopes of one target polypeptide or may contain antigen-binding domains specific for epitopes of more than one target polypeptide. An exemplary bi-specific antibody format that can be used in the context certain embodiments involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In various embodiments, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise an Y96F modification (by Y436F by EU). Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of certain embodiments. Any multispecific antibody format, including the exemplary bispecific antibody formats disclosed herein, may in various embodiments be adapted for use in the context of an antigen-binding fragment of an anti-IL-6R antibody using routine techniques available in the art.

The fully-human anti-IL-6R antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are back-mutated to the corresponding germline residue(s) or to a conservative amino acid substitution (natural or non-natural) of the corresponding germline residue(s) (such sequence changes are referred to herein as “germline back-mutations”). A person of ordinary skill in the art, starting with the heavy and light chain variable region sequences disclosed herein, can easily produce numerous antibodies and antigen-binding fragments which comprise one or more individual germline back-mutations or combinations thereof. In certain embodiments, all of the framework residues and/or CDR residues within the VH and/or VL domains are mutated back to the germline sequence. In various embodiments, only certain residues are mutated back to the germline sequence, e.g., only the mutated residues found within the first 8 amino acids of FR1 or within the last 8 amino acids of FR4, or only the mutated residues found within CDR1, CDR2 or CDR3. Furthermore, included herein are antibodies that may contain any combination of two or more germline back-mutations within the framework and/or CDR regions, i.e., wherein certain individual residues are mutated back to the germline sequence while certain other residues that differ from the germline sequence are maintained. Once obtained, antibodies and antigen-binding fragments that contain one or more germline back-mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies featured in the disclosure may in various embodiments nonetheless include amino acid residues not encoded by human germline immunoglobulin sequences (e.g., mutations introduced by random or site-specific mutagenesis in vitro or by somatic mutation in vivo), for example in the CDRs and in some embodiments CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al., (1992) Nucl. Acids Res. 20:6287-6295, incorporated herein by reference in its entirety,) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In an embodiment, an immunoglobulin molecule comprises a stable four chain construct of approximately 150-160 kDa in which the dimers are held together by an interchain heavy chain disulfide bond. In another embodiment, the dimers are not linked via inter-chain disulfide bonds and a molecule of about 75-80 kDa is formed composed of a covalently coupled light and heavy chain (half-antibody). These embodiments/forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al., (1993) Molecular Immunology 30:105, incorporated by reference in its entirety) to levels typically observed using a human IgG1 hinge. The instant disclosure encompasses in various embodiments antibodies having one or more mutations in the hinge, CH2 or CH3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” In various embodiments, the isolated antibody also includes an antibody in situ within a recombinant cell. In various embodiments, isolated antibodies are antibodies that have been subjected to at least one purification or isolation step. In various embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-6R, as used herein, includes antibodies that bind IL-6R (e.g., human IL-6R) or portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured in a surface plasmon resonance assay. In some embodiments, the antibody binds IL-6R (e.g., human IL-6Rα) with a KD of from about 0.1 nM to about 1000 nM or from about 1 nM to about 100 nM. In some embodiments, the antibody binds IL-6R (e.g., human IL-6Rα) with a KD of from about 1 pM to about 100 pM or from about 40 pM to about 60 pM. Specific binding can also be characterized by a dissociation constant of at least about 1×10⁻⁶ M or smaller. In various embodiments, the dissociation constant is at least about 1×10⁻⁷ M, 1×10⁻⁸ M, or 1×10⁻⁹ M. An isolated antibody that specifically binds human IL-6R may, however, have cross-reactivity to other antigens, such as IL-6R molecules from other (non-human) species.

The term “surface plasmon resonance”, as used herein, refers to an optical phenomenon that allows for the analysis of real-time interactions by detection of alterations in protein concentrations within a biosensor matrix, for example using the BIACORE system (Biacore Life Sciences division of GE Healthcare, Piscataway, N.J.).

The term “KD”, as used herein, is intended to refer to the equilibrium dissociation constant of an antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The anti-IL-6R antibodies useful for the methods described herein may in various embodiments include one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes in various embodiments methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). Numerous antibodies and antigen-binding fragments may be constructed which comprise one or more individual germline mutations or combinations thereof. In certain embodiments, all of the framework and/or CDR residues within the VH and/or VL domains are mutated back to the residues found in the original germline sequence from which the antibody was derived. In various embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In various embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a certain germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present disclosure.

The present disclosure also includes methods involving the use of anti-IL-6R antibodies comprising variants of any of the HCVR, LCVR, and/or CDR amino acid sequences disclosed herein having one or more conservative substitutions. For example, the present disclosure includes the use of anti-IL-6R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

According to the present disclosure, the anti-IL-6R antibody, or antigen-binding fragment thereof, in various embodiments comprises a heavy chain variable region (HCVR), light chain variable region (LCVR), and/or complementarity determining regions (CDRs) comprising any of the amino acid sequences of the anti-IL-6R antibodies described in U.S. Pat. No. 7,521,052, incorporated herein by reference in its entirety. The hybridoma cell line producing TCZ has been internationally deposited at International Patent Organism Depository (AIST Tsukuba Central 6, 1-1, Higashi 1-chome, Tsukuba-shi, Ibaraki Pref) on the basis of Budapest Treaty as FERM BP-2998 on Jul. 12, 1989. In certain embodiments, the anti-IL-6R antibody or antigen-binding fragment thereof comprises the heavy chain complementarity determining regions (HCDRs) and or the light chain complementarity determining regions (LCDRs) of a HCVR comprising the amino acid sequence of SEQ ID NO: 2 and the light chain complementarity determining regions (LCDRs) of a LCVR comprising the amino acid sequence of SEQ ID NO: 1. According to certain embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO: 6; the HCDR2 comprises the amino acid sequence of SEQ ID NO: 7; the HCDR3 comprises the amino acid sequence of SEQ ID NO: 8; the LCDR1 comprises the amino acid sequence of SEQ ID NO: 3; the LCDR2 comprises the amino acid sequence of SEQ ID NO: 4; and the LCDR3 comprises the amino acid sequence of SEQ ID NO: 5. In various embodiments, the anti-IL-6R antibody or antigen-binding fragment thereof comprises an heavy chain comprising the amino acid sequence of SEQ ID NO: 2 and an light chain comprising the amino acid sequence of SEQ ID NO: 1.

In another embodiment, the anti-IL-6R antibody or antigen-binding fragment thereof comprises a heavy chain comprising the amino acid sequence of the heavy chain of TCZ and a light chain comprising the amino acid sequence of the light chain of TCZ. In some embodiments, the extracellular domain of hIL-6R comprises the amino acid sequence of the extracellular domain of TCZ. According to certain exemplary embodiments, the methods of the present disclosure comprise the use of the anti-IL-6R antibody referred to and known in the art as tocilizumab, or a bioequivalent thereof.

The amino acid sequence of SEQ ID NO: 1 is

DIQMTQSPSSLSASVGDRVTITCRASQDISSYLNWYQQKPGKAPKLLIYY
TSRLHSGVPSRFSGSGSGTDFTFTISSLQPEDIATYYCQQGNTLPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The amino acid sequence of SEQ ID NO: 2 is,

VQLQESGPGLVRPSQTLSLTCTVSGYSITSDHAWSWVRQPPGRGLEWIGY
ISYSGITTYNPSLKSRVTMLRDTSKNQFSLRLSSVTAADTAVYYCARSLA
RTTAMDWGQGSLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG

The amino acid sequence of SEQ ID NO: 3 is, RASQDISSYLN

The amino acid sequence of SEQ ID NO: 4 is, YTSRLHS

The amino acid sequence of SEQ ID NO: 5 is, QQGNTLPYT

The amino acid sequence of SEQ ID NO: 6 is, SDHAWS

The amino acid sequence of SEQ ID NO: 7 is, YISYSGITTYNPSLK

The amino acid sequence of SEQ ID NO: 8 is, SLARTTAMDY

The term “bioequivalent” as used herein, refers to a molecule having similar bioavailability (rate and extent of availability) after administration at the same molar dose and under similar conditions (e.g., same route of administration), such that the effect, with respect to both efficacy and safety, can be expected to be essentially same as the comparator molecule. Two pharmaceutical compositions comprising an anti-IL-6R antibody are bioequivalent if they are pharmaceutically equivalent, meaning they contain the same amount of active ingredient (e.g., IL-6R antibody), in the same dosage form, for the same route of administration and meeting the same or comparable standards. Bioequivalence can be determined, for example, by an in vivo study comparing a pharmacokinetic parameter for the two compositions. Parameters commonly used in bioequivalence studies include peak plasma concentration (Cmax) and area under the plasma drug concentration time curve (AUC).

### DMARDs

Disease-modifying antirheumatic drugs (DMARDs) are drugs defined by their use in rheumatoid arthritis to slow down disease progression.

DMARDs have been classified as synthetic (sDMARD) and biological (bDMARD). Synthetic DMARDs include non-exhaustively methotrexate, sulfasalazine, leflunomide, and hydroxychloroquine. Biological DMARDs include non-exhaustively adalimumab, golimumab, etanercept, abatacept, infliximab, rituximab, and sarilumab.

### Methods of Administration and Formulations

The methods described herein comprise administering a therapeutically effective amount of an anti-IL-6R antibody to a subject. As used herein, an “effective amount” or “therapeutically effective amount” is a dose of the therapeutic that results in treatment of rheumatoid arthritis (RA). As used herein, “treating” refers to causing a detectable improvement in one or more symptoms associated with RA or causing a biological effect (e.g., a decrease in the level of a particular biomarker) that is correlated with the underlying pathologic mechanism(s) giving rise to the condition or symptom(s). For example, a dose of anti-IL-6R antibody which causes an improvement in any of the following symptoms or conditions associated with RA is deemed a “therapeutically effective amount”: tender joints, swollen joints, joint stiffness, fatigue, fever or loss of appetite.

In various embodiments, subjects with moderately-to-severely active rheumatoid arthritis have at least 6 of 66 swollen joints and 8 of 68 tender joints, as counted by the physician in a typical quantitative swollen and tender joint count examination and/or high sensitivity C-reactive protein (hs-CRP)≥8 mg/L or erythrocyte sedimentation rate (ESR)≥28 mm/H and/or Disease Activity Score 28—Erythrocyte Sedimentation Rate (DAS28ESR)≥5.1.

In various embodiments, a subject has a Disease Activity Score (DAS) of from 3.2 to 5.1. In various embodiments, a subject has a DAS of greater than 5.1. In various embodiments, the subject has a DAS of 3.2 or more. In various embodiments, the subject has a DAS of from 5 to 6, from 5 to 7, from 5 to 8, from 5 to 9, from 5 to 10, or from 7.5 to 10. The DAS for a subject can readily be calculated by those in the art. Non-limiting descriptions relating to DAS are provided in Fransen and van Riel (Clin Exp Rheumatol. 2005 September-October; 23 (5 Suppl 39):S93-9), the entire content of which is incorporated herein by reference.

An “improvement” in an RA-associated symptom in various embodiments refers reduction in the incidence of the RA symptom which may correlate with an improvement in one or more RA-associated test, score or metric (as described herein). In an embodiment, improvement may comprise a decrease in baseline of stiffness (e.g., a joint with limited motion). As used herein, the term “baseline,” with regard to an RA-associated parameter, means the numerical value of the RA-associated parameter for a patient prior to or at the time of administration of the antibody of the present invention. A detectable “improvement” can also be detected using at least one test, score or metric described herein. In various embodiments, the improvement is detected using at least one selected from the group consisting of: American College of Rheumatism (ACR), (e.g., ACR30, ACR50 and ACR70). In various embodiments, the improvement is characterized by at least one score or metric, such as physician global assessment of disease activity score, patient or parent assessment of overall well-being, number of joints with active arthritis, number of joints with limited motion, and/or high sensitivity C-reactive protein. In various embodiments, the improvement is characterized by at least one biomarker.

In another example, a treatment has not been effective when a dose of anti-IL-6R antibody does not result in a detectable improvement in one or more parameters or symptoms associated with RA or which does not cause a biological effect that is correlated with the underlying pathologic mechanism(s) giving rise to the condition or symptom(s) of RA.

According to some of these embodiments, the IL-6R antibody is administered subcutaneously. According to some of these embodiments, the IL-6R antibody is tocilizumab.

In accordance with some methods disclosed herein, a therapeutically effective amount of anti-IL-6R antibody that is administered to the subject varies depending upon the age and the size (e.g., body weight or body surface area) of the subject as well as the route of administration and other factors well known to those of ordinary skill in the art. In various embodiments, the dose varies based on the bodyweight of the subject.

In various embodiments, the dose of the antibody varies depending on the gender, age, or symptoms of a subject. In various embodiments, certain subject populations are selected based upon these criteria. In various embodiments, these selected subject populations are administered an escalated dose of antibody within 30 days of the beginning of treatment with the antibody. In various embodiments, the selected subjects are administered an escalated dose of antibody after they have been administered the antibody no more than 1, 2, 3, 4, 5, 6, 7, 8, 9, 10, 11, 12, 13, 14, 15, 16, 17, 18, 19, 20, 21, 22, 23, 24, 25, 26, 27, 28. 29, 30, 35, 40, 45, 50, 55 or 60 days. In various embodiments, selected subjects are administered an escalated dose of antibody when the antibody is first administered to the subject. In various embodiments, the selected subjects were not administered the antibody for more than 1, 2, 3, 4, 5, 6, 7, 8, 9, 10, 15, 20, 25 or 30 years before they are administered an escalated dose of the antibody.

In various embodiments, the antibody is administered at a non-escalated dose. In various embodiments, a non-escalated dose is less than 8 mg/kg administered intravenously every 4 weeks. In various embodiments, a non-escalated dose is about 8 mg/kg administered intravenously every 5, 6, 7, 8, 9, 10. 11, 12, 13, 14, 15 or 16 weeks. In various embodiments, a non-escalated dose is 8 mg/kg administered intravenously every 5, 6, 7, 8, 9, 10, 11, 12, 13, 14, 15 or 16 weeks. In various embodiments, a non-escalated dose is about 1.0, about 1.5, about 2.0, about 2.5, about 3.0, about 3.5, about 4.0, about 4.5, about 5.0, about 5.5, about 6.0, about 6.5, about 7.0 or about 7.5 mg/kg administered intravenously every four weeks. In various embodiments, a non-escalated dose is 1.0, 1.5, 2.0, 2.5, 3.0, 3.5, 4.0, 4.5, 5.0, 5.5, 6.0, 6.5, 7.0 or 7.5 mg/kg administered intravenously every four weeks. In various embodiments, a non-escalated dose is 2-6 mg/kg administered intravenously every four weeks. In various embodiments, a non-escalated dose is about 4 mg/kg administered intravenously every four weeks. In various embodiments, a non-escalated dose is 4 mg/kg administered intravenously every four weeks.

In various embodiments, a non-escalated dose is less than 162 mg administered subcutaneously every week. In various embodiments, a non-escalated dose is about 50, about 75, about 100, about 125, about 150 or about 162 mg administered subcutaneously every 2, 3, 4, 5, 6, 7, 8, 9 or 10 weeks. In various embodiments, a non-escalated dose is 50, 75, 100, 125, 150 or 162 mg administered subcutaneously every 2, 3, 4, 5, 6, 7, 8, 9 or 10 weeks. In various embodiments, a non-escalated dose is about 50, about 75, about 100, about 125 or about 150 mg administered subcutaneously every week. In various embodiments, a non-escalated dose is 50, 75, 100, 125 or 150 mg administered subcutaneously every week. In various embodiments, a non-escalated dose is about 162 mg administered subcutaneously every two weeks. In various embodiments, a non-escalated dose is 162 mg administered subcutaneously every two weeks.

In various embodiments, the antibody is administered an escalated dose. In various embodiments, an escalated dose is at least 8 mg/kg administered intravenously every 4 weeks. In various embodiments, an escalated dose is at least 4 mg/kg administered intravenously every 1, 2 or 3 weeks. In various embodiments, an escalated dose is at about 4, about 5, about 6, about 7, about 8, about 9, about 10, about 11, about 12, about 13, about 14, about 15 or about 16 mg/kg administered intravenously every 1, 2 or 3 weeks. In various embodiments, an escalated dose is at 4, 5, 6, 7, 8, 9, 10, 11, 12, 13, 14, 15 or 16 mg/kg administered intravenously every 1, 2 or 3 weeks. In various embodiments, an escalated dose is about 8, about 9, about 10, about 11, about 12, about 13, about 14, about 15 or about 16 mg/kg administered intravenously every four weeks. In various embodiments, an escalated dose is 8, 9, 10, 11, 12, 13, 14, 15 or 16 mg/kg administered intravenously every four weeks. In various embodiments, an escalated dose is about 8 mg/kg administered intravenously every four weeks. In various embodiments, an escalated dose is 8 mg/kg administered intravenously every four weeks.

In various embodiments, an escalated dose is at least 162 mg administered subcutaneously every week. In various embodiments, an escalated dose is about 162, about 175, about 200, about 225, about 250, about 275 or about 300 mg administered subcutaneously every week. In various embodiments, an escalated dose is 162, 175, 200, 225, 250, 275 or 300 mg administered subcutaneously every week. In various embodiments, an escalated dose is about 175, about 200, about 225, about 250, about 275 or about 300 mg administered subcutaneously every two weeks. In various embodiments, an escalated dose is 175, 200, 225, 250, 275 or 300 mg administered subcutaneously every two weeks. In various embodiments, an escalated dose is about 162 mg administered subcutaneously every week. In various embodiments, an escalated dose is 162 mg administered subcutaneously every week.

In various embodiments, subjects and subject populations are selected for administration of an escalated dose of antibody as described above. In various embodiments, subjects or subject populations are selected on the basis of gender. In various embodiments, females are selected for administration of an escalated dose of antibody as described above. In various embodiments, subjects or subject populations are selected on the basis of their age. In various embodiments, subjects from 18 to 34 years of age are selected for administration of an escalated dose of antibody as described above.

In embodiments, subjects or subject populations are selected on the basis of drugs that are being or not being administered to the subjects or subject populations. In various embodiments, subjects who have not been administered a corticosteroid within 90 days are selected for administration of an escalated dose of antibody as described above. In various embodiments, subjects who have not been administered a corticosteroid within 10, 20, 30, 40, 50, 60, 70, 80, 90, 100, 110 or 120 days are selected for administration of an escalated dose of antibody as described above. In various embodiments, corticosteroids include bethamethasone, prednisone, prednisolone, triamcinolone, methylprednisolone or dexamethasone. In various embodiments, the corticosteroid is prednisone.

In various embodiments, subjects or subject populations are selected on the basis of certain symptoms or pathologies they have or are absent. In various embodiments, subjects without anemia are selected for administration of an escalated dose of antibody as described above. In various embodiments, subjects with depression are selected for administration of an escalated dose of antibody as described above.

In various embodiments, anemia includes diseases associated with iron deficiency and iron maldistribution. In various embodiments, anemia includes anemia of chronic disease, anemia of inflammation, iron deficiency anemia, functional iron deficiency, and microcytic anemia. The terms “anemia of chronic disease” or “anemia of inflammation” refer to any anemia that develops as a result of, for example, extended infection, inflammation, neoplastic disorders, etc. Without being bound by any scientific theory, the anemia which develops is often characterized by a shortened red blood cell life span and sequestration of iron in macrophages, which results in a decrease in the amount of iron available to make new red blood cells.

In various embodiments, depression includes minor and major depression. Symptoms of depression include anhedonia, low mood, changes in sleep, appetite, energy level, concentration, daily behavior, or self-esteem.

In various embodiments, a selected subject or subject population has one or more of the traits described above, i.e., a selected subject can be a female, be 18-34 years of age, not have anemia, have depression, and/or have not used a corticosteroid in a number of days as described above.

Various delivery systems are known and can be used to administer the pharmaceutical composition described herein, e.g., encapsulation in liposomes, microparticles, microcapsules, receptor mediated endocytosis (see, e.g., Wu et al. (1987) J. Biol. Chem. 262:4429-4432, incorporated herein by reference in its entirety). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The IL-6R antibody can be administered subcutaneously or intravenously.

The pharmaceutical composition can also be delivered in a vesicle, such as a liposome (see Langer (1990) Science 249:1527-1533, incorporated herein by reference in its entirety). In certain situations, the pharmaceutical composition can be delivered in a controlled release system, for example, with the use of a pump or polymeric materials. In another embodiment, a controlled release system can be placed in proximity of the composition's target, thus requiring only a fraction of the systemic dose.

The injectable preparations may include dosage forms for intravenous, subcutaneous, intracutaneous and intramuscular injections, local injection, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc.). As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

### EXAMPLE

The aim of the current study is to understand real-world dose modification patterns of SC TCZ among RA patients from the United States. The study retrospectively examined the starting dose of SC TCZ among RA patients who initiated therapy with SC TCZ, the frequency of SC TCZ dose modifications during 1-year follow-up, time to dose modification, and predictors of dose escalation.

### Baseline Characteristics

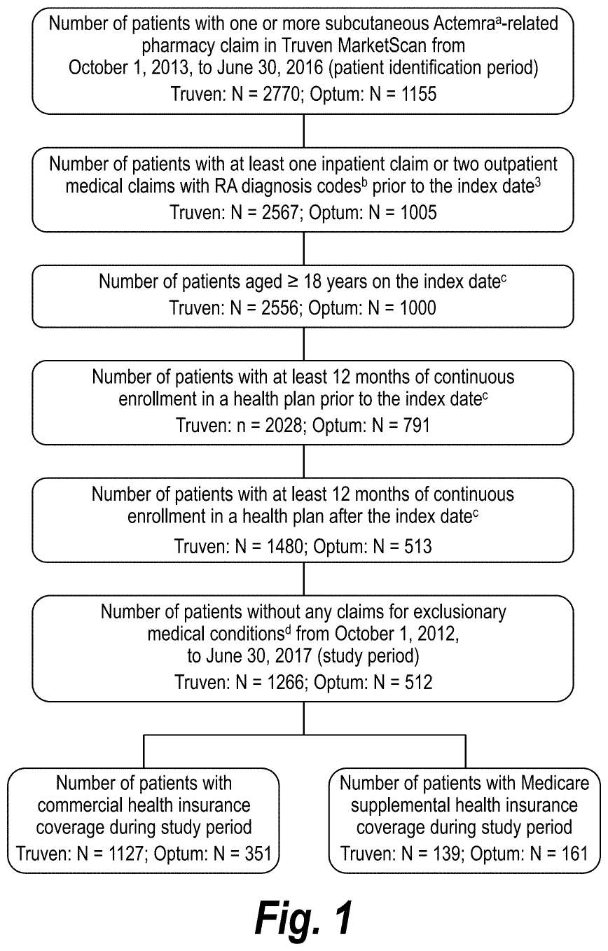

The study sample included data from 1266 patients in the Truven MarketScan database and 512 patients in the Optum Clinformatics database between Oct. 1, 2012, and Jun. 30, 2017 (study period) (FIG. 1). Adults meeting the inclusion criteria between Oct. 1, 2013 and Jun. 30, 2016 (patient identification period) were included in the study sample. The first fill date of subcutaneous (SC) tocilizumab (TCZ) during the patient identification period was the index date. The primary grouping variables used in the study were Medicare and Commercial, and patients with Medicare Supplemental coverage during the entire study period were included in the ‘Medicare’ group, while the remaining patients were included in the ‘Commercial’ group.

### Inclusion and Exclusion Criteria

Patients were included if they had ≥1 pharmacy claim for SC TCZ during the patient identification period; had >1 inpatient or >2 outpatient medical claims with RA diagnosis codes (International Classification of Diseases [ICD]-9: 714.XX; ICD 10: M05.XX or M06.XX) before the index date; were aged >18 years on the index date; and had >12 months continuous enrollment in a commercial health plan before and after the index date (baseline and follow-up periods, respectively).

Patients with ≥1 medical claims during the study related to the following diagnoses were excluded: ankylosing spondylitis (ICD-9: 720.0x; ICD-10: M08.1, M45.xx), Crohn's disease (ICD-9: 555.xxx; ICD-10: K50.00), juvenile idiopathic arthritis (ICD 9: 714.3x; ICD-10: M08.xx), psoriasis (ICD-9: 696.1x; ICD-10: L40.x), psoriatic arthritis (ICD-9: 696.xx; ICD-10: L40.xx), ulcerative colitis (ICD-9: 556.xx: ICD-10: K51.xx), chronic lymphocytic leukemia (ICD-9: 204.1x; ICD-10: C91.10), non-Hodgkin's lymphoma (ICD-9: 202.8x; ICD-10: C85.90), or giant-cell arteritis (ICD-9: 446.5x; ICD-10:M13.6x).

Study Endpoints

The average monthly dose (AMD) of SC TCZ was calculated as the quantity dispensed×strength/days of supply×28.

The following dose categories of SC TCZ were used in the study: <324 mg/28 days (initiated at a lower dose than outlined in the label); 324 mg/28 days (i.e., 162 mg Q2W; recommended starting dose for patients weighing <100 kg); between 324 mg/28 days and 648 mg/28 days; 648 mg/28 days (i.e., 162 mg QW; recommended starting dose for patients weighing >100 kg or escalated dose for patients weighing <100 kg); and >648 mg/28 days (higher dose than recommended in the product label).

The following demographic and clinical characteristics were assessed during the baseline period for the study sample: age on the index date, gender, region of patients' residence, comorbid conditions, Elixhauser comorbidity index (ECI) score, and previous RA treatment (csDMARDs and biologics). Index therapy, including type of index therapy (monotherapy or combination therapy), and index dose were assessed on the index date or plus 90 days from the index date.

The number of SC TCZ fills per 28 days was calculated using distinct fill dates associated with SC TCZ. Dose escalation was defined as an index AMD of 324 mg/28 days, followed by an AMD of 648 mg/28 days after the index date. Dose reduction was defined as an index AMD of 648 mg/28 days, then an AMD of 324 mg/28 days after the index date.

Time to first dose escalation was the number of days between the index date and first fill of SC TCZ at an escalated dose. Time to first dose reduction was the number of days between the index date and first fill of SC TCZ at a reduced dose.

During the follow-up period, the number of days the patient was covered by SC TCZ was counted, based on the prescription fill date and the number of days of supply. If the number of days of supply for SC TCZ prescriptions overlapped, then the prescription start date of the second fill was adjusted to the day after the previous fill ended. This helped to consider non-overlapping days covered by SC TCZ prescriptions. To calculate the proportion of days covered as a percentage for each patient, the number of days covered was divided by the number of days in the follow up period (365 in this study) and multiplied by 100.

### Statistical Analysis

Descriptive analyses were conducted for all study outcomes, and descriptive statistics for all study outcomes were reported for the overall study sample as well as by primary grouping variables (Medicare and Commercial). Mean, standard deviation (SD), and median values were reported for continuous variables, and frequency (N and percentage) was reported for categorical variables.

Time to first dose modification (escalation and reduction) was analyzed using Kaplan-Meier analysis for those patients with a dose modification.

A logistic regression model that included primary grouping variables, index therapy (monotherapy SC TCZ vs SC TCZ/csDMARD combination therapy), and baseline patient characteristics was used to identify predictors of likelihood of dose escalation in the study sample. A Cox proportional hazards regression model, that included primary grouping variables, index therapy (monotherapy vs combination therapy), and baseline patient characteristics, was used to identify predictors of time to dose escalation among patients who escalated.

### Results

Baseline Characteristics

The mean (SD) age was 52.3 (±10.7) years for Truven and 54.9 (±13.3) years for Optum patients; the proportion of females was 82% in Truven and 83% in Optum; mean (SD) follow-up was 25.8 (±9.2) months for Truven and 27.9 (±9.1) months for Optum patients; and mean (SD) ECI score was 1.8 (±1.9) for Truven and 2.3 (±2.4) for Optum patients (Table 1). Patients in the Truven and Optum cohorts with Commercial and Medicare coverage, respectively, had a mean (SD) age of 50.3 (9.2) and 69.1 (6.6) (Truven) and 50.3 (11.9) and 64.7 (10.3) years (Optum); the proportion of females was 83% and 70% (Truven) and 80% and 89% (Optum); and mean (SD) ECI score was 1.7 (1.8) and 2.8 (2.3) (Truven) and 1.7 (7.7) and 3.9 (2.9).

Twelve months before the index date, csDMARDs, biologics, and corticosteroids were all commonly used among Truven and Optum patients (Truven: 72%, 75%, and 74%; and Optum: 71%, 71%, and 79%, respectively; Table 1). Baseline RA treatment patterns by coverage among Truven and Optum patients are also shown in Table 1.

TABLE 1
Baseline Demographic and Clinical Characteristics, and Treatment Patterns

| | Truven MarketScan | | | | | | Optum Clinformatics | | | | | |
|---|---|---|---|---|---|---|---|---|---|---|---|---|
| | Commercial | | Medicare | | Overall | | Commercial | | Medicare | | Overall | |
| Characteristics | (N = 1127) | | (N = 139) | | (N = 1266) | | (N = 351) | | (N = 161) | | (N = 512) | |
| Age on the index date, | 50.3 | (9.2) | 69.1 | (6.6) | 52.3 | (10.7) | 50.3 | (11.9) | 64.7 | (10.3) | 54.9 | (13.3) |
| mean, years (SD) | | | | | | | | | | | | |
| Female, N (%) | 939 | (83) | 97 | (70) | 1036 | (82) | 282 | (80) | 143 | (89) | 425 | (83) |
| Region, N (%) | | | | | | | | | | | | |
| North central | 184 | (16) | 35 | (25) | 219 | (17) | 73 | (21) | 20 | (12) | 93 | (18) |
| Northeast | 167 | (15) | 43 | (31) | 210 | (17) | 22 | (6) | 18 | (11) | 40 | (8) |
| South | 567 | (50) | 45 | (32) | 612 | (48) | 185 | (53) | 76 | (47) | 261 | (51) |
| West | 197 | (17) | 15 | (11) | 212 | (17) | 71 | (20) | 48 | (30) | 119 | (23) |
| Unknown | 12 | (1) | 1 | (1) | 13 | (1) | 0 | (0) | 1 | (1) | 1 | (0) |
| Follow-up duration, | 26.0 | (9.3) | 24.4 | (9.1) | 25.8 | (9.2) | 27.9 | (9.0) | 28.0 | (9.3) | 27.9 | (9.1) |
| mean, months (SD) | | | | | | | | | | | | |
| Index year, N (%) | | | | | | | | | | | | |
| 2013 | 60 | (5) | 10 | (7) | 70 | (6) | 6 | (2) | 8 | (5) | 14 | (3) |
| 2014 | 478 | (42) | 49 | (35) | 527 | (42) | 164 | (47) | 53 | (33) | 217 | (42) |
| 2015 | 417 | (37) | 57 | (41) | 474 | (37) | 132 | (38) | 55 | (34) | 187 | (37) |
| 2016 | 172 | (15) | 23 | (17) | 195 | (15) | 49 | (14) | 45 | (28) | 94 | (18) |
| ECI score, mean (SD) | 1.7 | (1.8) | 2.8 | (2.3) | 1.8 | (1.9) | 1.7 | (1.7) | 3.8 | (2.9) | 2.3 | (2.4) |
| ECI group, N (%) | | | | | | | | | | | | |
| 0 | 341 | (30) | 22 | (16) | 363 | (29) | 104 | (30) | 15 | (9) | 119 | (23) |
| 1 | 304 | (27) | 21 | (15) | 325 | (26) | 100 | (28) | 24 | (15) | 124 | (24) |
| 2 | 202 | (18) | 33 | (24) | 235 | (19) | 61 | (17) | 25 | (16) | 86 | (17) |
| ≥3 | 280 | (25) | 63 | (45) | 343 | (27) | 86 | (25) | 97 | (60) | 183 | (36) |
| Baseline RA treatment | | | | | | | | | | | | |
| patterns | | | | | | | | | | | | |
| csDMARDs^{a} | 814 | (72) | 103 | (74) | 917 | (72) | 244 | (70) | 118 | (73) | 362 | (71) |
| Biologics^{b} | 857 | (76) | 98 | (71) | 955 | (75) | 249 | (71) | 116 | (72) | 365 | (71) |
| Corticosteroids^{c} | 828 | (73) | 104 | (75) | 932 | (74) | 267 | (76) | 136 | (84) | 403 | (79) |

^{a}csDMARDs include hydroxychloroquine sulfate, leflunomide, methotrexate, and sulfasalazine.
^{b}Biologics include tumor necrosis factor inhibitors (certolizumab, etanercept, golimumab, adalimumab, and infliximab) and non-tumor necrosis factor inhibitors (abatacept, rituximab, tofacitinib, and intravenous tocilizumab).
^{c}Corticosteroids include prednisone, dexamethasone, hydrocortisone, methylprednisolone, prednisolone, triamcinolone, cortisone acetate, and betamethasone.
csDMARD indicates conventional synthetic disease-modifying antirheumatic drug; ECI, Elixhauser comoibidity index; RA, rheumatoid arthritis; SD, standard deviation.

Treatment Patterns

In the study sample, 90 days before the index date, 22% of Truven patients and 25% of Optum patients were without therapy; 47% each of Truven and Optum patients were receiving monotherapy; 31% of Truven patients and 29% of Optum patients received combination treatment with csDMARDs and biologics; and 51% of Truven patients and 57% of Optum patients had used corticosteroids. Among Truven and Optum patients with Commercial and Medicare coverage, respectively, 47% and 44% (Truven) and 46% and 48% (Optum) received monotherapy, and 30% and 35% (Truven) and 29% and 27% (Optum) received combination treatment with csDMARDS and biologics (Table 2).

TABLE 2
Proximal RA Treatment Patterns

Proximal RA treatment

| (90 days prior to the | Truven MarketScan | | | | | | Optum Clinformatics | | | | | |
|---|---|---|---|---|---|---|---|---|---|---|---|---|
| index date but closest to | Commercial | | Medicare | | Overall | | Commercial | | Medicare | | Overall | |
| the index date), N (%) | (N = 1127) | | (N = 139) | | (N = 1266) | | (N = 351) | | (N = 161) | | (N = 512) | |
| Without therapy | 253 | (22) | 30 | (22) | 283 | (22) | 86 | (25) | 40 | (25) | 126 | (25) |
| Monotherapy | 535 | (47) | 61 | (44) | 596 | (47) | 163 | (46) | 77 | (48) | 240 | (47) |
| Only csDMARDs | 275 | (24) | 36 | (26) | 311 | (25) | 78 | (22) | 47 | (29) | 125 | (24) |
| Hydroxychloroquine | 75 | (7) | 13 | (9) | 88 | (7) | 13 | (4) | 12 | (7) | 25 | (5) |
| sulfate | | | | | | | | | | | | |
| Leflunomide | 53 | (5) | 10 | (7) | 63 | (5) | 17 | (5) | 8 | (5) | 25 | (5) |
| Methotrexate | 173 | (15) | 18 | (13) | 191 | (15) | 46 | (13) | 32 | (20) | 78 | (15) |
| Sulfasalazine | 24 | (2) | 3 | (4) | 29 | (2) | 13 | (4) | 7 | (4) | 20 | (4) |
| Only biologies | 260 | (33) | 25 | (18) | 285 | (23) | 85 | (24) | 30 | (19) | 115 | (22) |
| TNFi | 158 | (14) | 15 | (11) | 173 | (14) | 57 | (16) | 15 | (9) | 72 | (14) |
| Certolizumab | 22 | (2) | 0 | (0) | 22 | (2) | 13 | (4) | 2 | (1) | 15 | (3) |
| Etanercept | 64 | (6) | 5 | (4) | 69 | (5) | 20 | (6) | 5 | (3) | 25 | (5) |
| Golimumab | 10 | (1) | 1 | (1) | 11 | (1) | 8 | (2) | 3 | (2) | 11 | (2) |
| Adalimumab | 62 | (6) | 9 | (6) | 71 | (6) | 19 | (5) | 5 | (3) | 24 | (5) |
| Infliximab | 1 | (0) | 0 | (0) | 1 | (0) | 0 | (0) | 0 | (0) | 0 | (0) |
| Abatacept | 50 | (4) | 7 | (5) | 57 | (5) | 14 | (4) | 11 | (7) | 25 | (5) |
| Rituximab | 1 | (0) | 0 | (0) | 1 | (0) | 0 | (0) | 0 | (0) | 0 | (0) |
| Tofacitinib | 40 | (4) | 5 | (4) | 45 | (4) | 14 | (4) | 5 | (3) | 19 | (4) |
| IV TCZ | 53 | (5) | 5 | (4) | 58 | (5) | 15 | (4) | 5 | (3) | 20 | (4) |
| Combination therapy | | | | | | | | | | | | |
| csDMARDs + biologies | 339 | (30) | 48 | (35) | 387 | (31) | 102 | (29) | 44 | (27) | 146 | (29) |
| Corticosteroid use (+90 | 576 | (51) | 72 | (52) | 648 | (51) | 189 | (54) | 105 | (65) | 294 | (57) |
| days prior to the index | | | | | | | | | | | | |
| date) | | | | | | | | | | | | |
| Prednisone | 498 | (44) | 64 | (46) | 562 | (44) | 158 | (45) | 94 | (58) | 252 | (49) |
| Dexamethasone | 5 | (0) | 0 | (0) | 5 | (0) | 1 | (0) | 2 | (1) | 3 | (1) |
| Hydrocortisone | 4 | (0) | 1 | (1) | 5 | (0) | 1 | (0) | 1 | (1) | 2 | (0) |
| Methylprednisolone | 101 | (9) | 16 | (12) | 117 | (9) | 36 | (10) | 13 | (8) | 49 | (10) |
| Prednisolone | 1 | (0) | 0 | (0) | 1 | (0) | 0 | (0) | 0 | (0) | 0 | (0) |
| Triamcinolone | 14 | (1) | 2 | (1) | 16 | (1) | 0 | (0) | 5 | (3) | 5 | (1) |
| Cortisone acetate | 0 | (0) | 0 | (0) | 0 | (0) | 0 | (0) | 0 | (0) | 0 | (0) |
| Betamethasone | 5 | (0) | 0 | (0) | 5 | (0) | 1 | (0) | 0 | (0) | 1 | (0) |

csDMARD indicates conventional synthetic disease-modifying antirheumatic drag; IV, intravenous; RA, rheumatoid arthritis; TCZ, tocilizumab; TNFi, tumor necrosis factor inhibitor.

Approximately half of the patients initiated SC TCZ as monotherapy (Truven, 44%; and Optum, 47%), while the other half initiated SC TCZ as combination therapy (Truven, 56%; and Optum, 53%). Among Truven and Optum patients with Commercial and Medicare coverage, respectively, 44% and 39% (Truven) and 50% and 42% (Optum) initiated SC TCZ as monotherapy, while 56% and 61% (Truven), and 50% and 58% (Optum) initiated SC TCZ as combination therapy. Among patients who initiated SC TCZ with csDMARDs (Truven, 49%; and Optum, 48%), methotrexate was the most commonly used csDMARD (Truven, 32%; and Optum, 30%). A small proportion of patients initiated SC TCZ with another bDMARD (Truven, 3%; and Optum, 1%) (Table 3).

TABLE 3
Subcutaneous Tocilizumab Index Therapy, Dose, Fills, and Dose Modifications

| | Truven MarketScan | | | | | | Optum Clinformatics | | | | | |
|---|---|---|---|---|---|---|---|---|---|---|---|---|
| Index therapy and | Commercial | | Medicare | | Overall | | Commercial | | Medicare | | Overall | |
| dose, N (%) | (N = 1127) | | (N = 139) | | (N = 1266) | | (N = 351) | | (N = 161) | | (N = 512) | |
| Monotherapy | 499 | (44) | 54 | (39) | 553 | (44) | 174 | (50) | 67 | (42) | 241 | (47) |
| Combination therapy | 628 | (56) | 85 | (61) | 713 | (56) | 177 | (50) | 94 | (58) | 271 | (53) |
| (index date | | | | | | | | | | | | |
| [inclusive] +90 | | | | | | | | | | | | |
| days) with biologies | | | | | | | | | | | | |
| or csDMARDs | | | | | | | | | | | | |
| SC TCZ + | 546 | (48) | 75 | (54) | 621 | (49) | 163 | (46) | 81 | (50) | 244 | (48) |
| csDMARDs | | | | | | | | | | | | |
| Hydroxychloroquine | 150 | (13) | 20 | (14) | 170 | (13) | 32 | (9) | 18 | (11) | 50 | (10) |
| sulfate | | | | | | | | | | | | |
| Leflunomide | 104 | (9) | 13 | (9) | 117 | (9) | 28 | (8) | 16 | (10) | 44 | (9) |
| Methotrexate | 363 | (32) | 48 | (35) | 411 | (32) | 104 | (30) | 50 | (31) | 154 | (30) |
| Sulfasalazine | 41 | (4) | 4 | (3) | 45 | (4) | 15 | (4) | 10 | (6) | 25 | (5) |
| SC TCZ + biologies | 39 | (3) | 4 | (3) | 43 | (3) | 0 | (0) | 5 | (3) | 5 | (1) |
| TNFi | 28 | (2) | 3 | (2) | 31 | (2) | 0 | (0) | 1 | (1) | 8 | (2) |
| Certolizumab | 6 | (1) | 0 | (0) | 6 | (0) | 0 | (0) | 0 | (0) | 1 | (0) |
| Etanercept | 9 | (1) | 2 | (1) | 11 | (1) | 0 | (0) | 1 | (1) | 3 | (1) |
| Golimumab | 4 | (0) | 0 | (0) | 4 | (0) | 0 | (0) | 0 | (0) | 0 | (0) |
| Adalimumab | 8 | (1) | 1 | (1) | 9 | (1) | 0 | (0) | 0 | (0) | 4 | (1) |
| Infliximab | 1 | (0) | 0 | (0) | 1 | (0) | 0 | (0) | 0 | (0) | 0 | (0) |
| Abatacept | 3 | (0) | 1 | (1) | 4 | (0) | 0 | (0) | 2 | (1) | 5 | (1) |
| Rituximab | 0 | (0) | 0 | (0) | 0 | (0) | 0 | (0) | 0 | (0) | 0 | (0) |
| Tofacitinib | 0 | (0) | 0 | (0) | 0 | (0) | 0 | (0) | 0 | (0) | 0 | (0) |
| IV TCZ | 8 | (1) | 0 | (0) | 8 | (1) | 0 | (0) | 3 | (2) | 5 | (1) |
| SC TCZ + | 43 | (4) | 6 | (4) | 49 | (4) | 14 | (4) | 8 | (5) | 22 | (4) |
| csDMARDs + | | | | | | | | | | | | |
| biologies | | | | | | | | | | | | |
| Corticosteroid use | 539 | (48) | 72 | (52) | 611 | (48) | 171 | (49) | 96 | (60) | 267 | (52) |
| (index date | | | | | | | | | | | | |
| [inclusive] +90 | | | | | | | | | | | | |
| days) | | | | | | | | | | | | |
| Prednisone | 444 | (39) | 63 | (45) | 507 | (40) | 139 | (40) | 84 | (52) | 223 | (44) |
| Dexamethasone | 2 | (0) | 0 | (0) | 2 | (0) | 1 | (0) | 4 | (2) | 5 | (1) |
| Hydrocortisone | 7 | (1) | 0 | (0) | 7 | (1) | 1 | (0) | 0 | (0) | 1 | (0) |
| Methylprednisolone | 110 | (10) | 14 | (10) | 124 | (10) | 27 | (8) | 13 | (8) | 40 | (8) |
| Prednisolone | 1 | (0) | 0 | (0) | 1 | (0) | 0 | (0) | 0 | (0) | 0 | (0) |
| Triamcinolone | 28 | (2) | 4 | (3) | 32 | (3) | 11 | (3) | 4 | (2) | 15 | (3) |
| Cortisone acetate | 0 | (0) | 0 | (0) | 0 | (0) | 0 | (0) | 0 | (0) | 0 | (0) |
| Betamethasone | 3 | (0) | 1 | (1) | 4 | (0) | 1 | (0) | 1 | (1) | 2 | (0) |
| SC TCZ index dose, | | | | | | | | | | | | |
| N (%) | | | | | | | | | | | | |
| <324 mg/28 days | 68 | (6) | 3 | (2) | 71 | (6) | 4 | (1) | 7 | (4) | 11 | (2) |
| 324 mg/28 days (ie, | 533 | (47) | 75 | (54) | 608 | (48) | 168 | (48) | 77 | (48) | 245 | (48) |
| 162 mg Q2W) | | | | | | | | | | | | |
| Between 324 and | 43 | (4) | 4 | (3) | 47 | (4) | 4 | (1) | 1 | (1) | 5 | (1) |
| 648 mg/28 days | | | | | | | | | | | | |
| 648 mg/28 days (ie, | 477 | (42) | 54 | (39) | 531 | (42) | 174 | (50) | 76 | (47) | 250 | (49) |
| 162 mg QW) | | | | | | | | | | | | |
| >648 mg/28 days | 5 | (0) | 0 | (0) | 5 | (0) | 1 | (0) | 0 | (0) | 1 | (0) |
| Missing dose | 1 | (0) | 3 | (2) | 4 | (0) | 0 | (0) | 0 | (0) | 0 | (0) |
| No. of SC TCZ fills/28 | 6.8 | (4.3) | 6.0 | (3.9) | 6.7 | (4.3) | 7.0 | (4.4) | 7.3 | (4.8) | 7.1 | (4.5) |
| days during follow-up | | | | | | | | | | | | |
| period, mean (SD) | | | | | | | | | | | | |
| Dose modifications | | | | | | | | | | | | |
| Index therapy with 324 | 533 | (47) | 75 | (55) | 608 | (48) | 168 | (48) | 77 | (48) | 245 | (48) |
| mg/28 days (ie, 162 mg | | | | | | | | | | | | |
| Q2W) | | | | | | | | | | | | |
| Dose escalation | 204 | (38) | 19 | (25) | 223 | (37) | 73 | (43) | 24 | (31) | 97 | (40) |
| Index therapy with 648 | 477 | (42) | 54 | (40) | 531 | (42) | 174 | (50) | 76 | (47) | 250 | (49) |
| mg/28 days (ie, 162 mg | | | | | | | | | | | | |
| QW) | | | | | | | | | | | | |
| Dose reduction | 14 | (0) | 3 | (6) | 17 | (3) | 5 | (3) | 4 | (5) | 9 | (4) |
| Proportion of days | 0.5 | (0.3) | 0.5 | (0.3) | 0.5 | (0.3) | 0.4 | (0.3) | 0.5 | (0.3) | 0.4 | (0.3) |
| covered, mean (SD) | | | | | | | | | | | | |

Most patients started with one of the recommended doses of SC TCZ 162 mg Q2W (Truven, 48%; and Optum, 48%) or 162 mg QW (Truven, 42%; and Optum, 49%). The remaining patients (Truven, 10%; and Optum, 3%) either initiated at a lower dose than outlined in the label (<324 mg/28 days) or were between the 162 mg Q2W and QW dose categories (324 mg/28 days and 648 mg/28 days) (Table 3).

Dose Modifications

During the 1-year follow-up period, of the patients who started on the 162 mg Q2W dose of SC TCZ, 37% from Truven and 40% from Optum escalated to 162 mg QW. Among patients who started on the 162 mg QW dose of SC TCZ, only 3% (Truven) and 4% (Optum) had a dose reduction to 162 mg Q2W (Table 2). Overall, 60% and 68% of patients in Truven and Optum initiated or escalated to the higher weekly dose. Among Truven and Optum patients with Commercial and Medicare coverage, respectively, 60% and 53% (Truven) and 70% and 62% (Optum) initiated or escalated to the higher weekly dose, while 0% and 6% (Truven) and 3% and 5% (Optum) had a dose reduction from 162 mg QW to 162 mg Q2W. The mean (SD) number of SC TCZ fills per 28 days during the follow-up period was 6.7 (±4.3) for Truven patients and 7.1 (+4.5) for Optum patients (Table 3). The mean (SD) proportion of days covered in the study sample was around 50% (Truven, 0.5 [±0.3]; and Optum, 0.4 [±0.3]; Table 3).

Time to Dose Increase

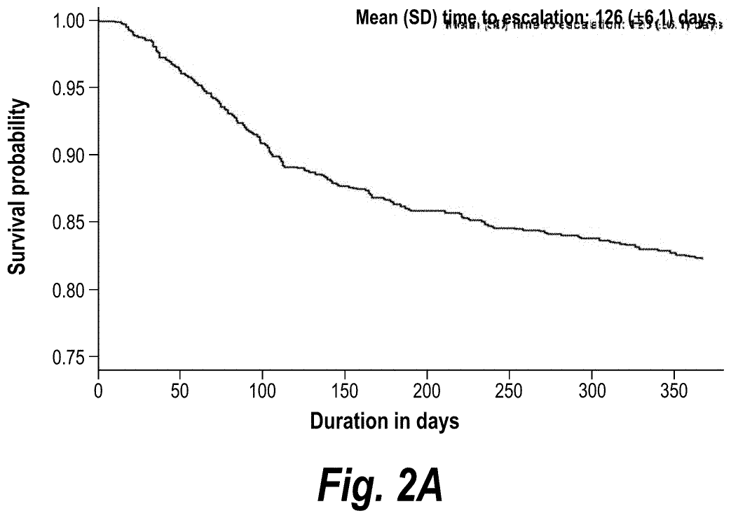

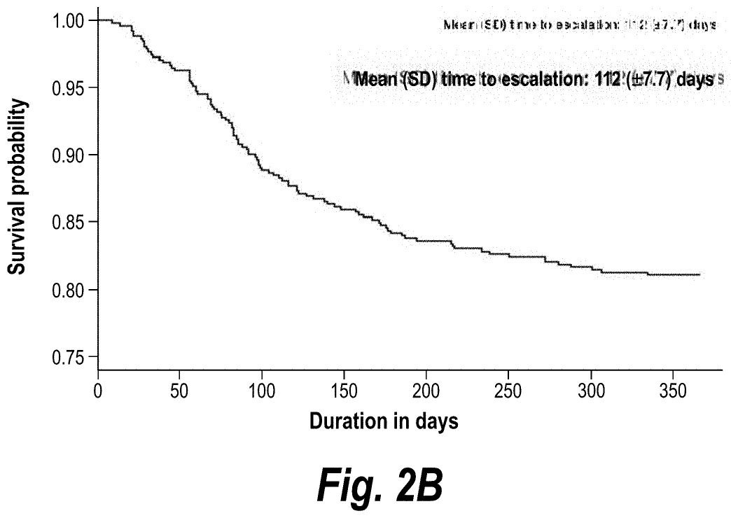

Among patients who had dose escalation, the mean (SD) time to dose escalation was 126 (±6.1) days for Truven patients and 112 (±7.7) days for Optum patients (FIGS. 2A and 2B).

Logistic Regression for Likelihood of Dose Escalation

Among Truven patients, corticosteroid use, age, and anemia (defined using ICD-9/ICD-10 diagnosis codes) were the three main predictors for dose escalation. Corticosteroid use within 90 days from the index date (odds ratio [OR]: 0.70; P=0.02), patients aged 35-44 years versus patients aged 18-34 years (OR: 0.54; P=0.05), or patients with anemia versus no anemia (OR: 0.50; P=0.04) had reduced odds of dose escalation (Table 4).

TABLE 4
Logistic Regression for Likelihood of First Dose Escalation
in Truven and Optum Patients Who Escalated Dose

| | | Odds | 95% hazard ratio | | P |
|---|---|---|---|---|---|
| Parameter | Reference | ratio | confidence limits | | value |
| Truven MarketScan | | | | | |
| Index combination | Yes vs no | 1.11 | −0.21 | 0.39 | .50 |
| therapy | | | | | |
| Corticosteroid use | | 0.70 | −0.65 | −0.05 | .02 |
| Commercial | Medicare | 1.09 | −0.80 | 1.12 | .86 |
| Age, years | | | | | |
| 35-44 | 18-34 years | 0.54 | −1.23 | 0.01 | .05 |
| 45-54 | | 0.59 | −1.08 | 0.04 | .06 |
| 55-64 | | 0.79 | −0.78 | 0.33 | .40 |
| Gender, female | Male | 1.10 | −0.30 | 0.52 | .64 |
| Geographical region | | | | | |
| North central | South | 1.19 | −0.25 | 0.59 | .41 |
| Northeast | | 0.94 | −0.52 | 0.37 | .77 |
| West | | 1.14 | −0.28 | 0.54 | .52 |
| Unknown | | 1.96 | −0.68 | 1.85 | .29 |
| ECI score | | | | | |
| 1 | ECI = 0 | 1.32 | −0.15 | 0.70 | .20 |
| 2 | | 1.32 | −0.27 | 0.81 | .31 |
| ≥3 | | 0.89 | −0.82 | 0.58 | .76 |
| Index year | | | | | |
| 2013 | 2016 | 1.80 | −0.08 | 1.25 | .08 |
| 2014 | | 1.00 | −0.44 | 0.47 | .99 |
| 2015 | | 0.93 | −0.52 | 0.40 | .77 |
| Comorbid conditions | | | | | |
| Diabetes | Yes vs no | 1.63 | −0.09 | 1.11 | .11 |
| CVD | | 0.85 | −0.61 | 0.30 | .47 |
| Hypertension | | 1.23 | −0.20 | 0.61 | .32 |
| Cancer | | 1.24 | −0.65 | 1.25 | .65 |
| Asthma | | 1.01 | −0.86 | 0.86 | .98 |
| COPD | | 1.18 | −0.48 | 0.89 | .62 |
| Anemia | | 0.50 | −1.32 | −0.02 | .04 |
| Rheumatoid vasculitis | | 0.32 | −2.62 | 0.48 | .13 |
| Osteoporosis | | 0.84 | −0.53 | 0.20 | .36 |
| Depression | | 0.89 | −0.71 | 0.48 | .70 |
| Mental illness | | 1.29 | −0.18 | 0.72 | .26 |
| Optum | | | | | |
| Index combination | Yes vs no | 1.46 | −0.19 | 0.93 | .19 |
| therapy | | | | | |
| Corticosteroid use | | 0.70 | −1.09 | 0.31 | .32 |
| Commercial | Medicare | 1.88 | −0.06 | 1.36 | .08 |
| Age, years | | | | | |
| 35-44 | 18-34 years | 0.85 | −1.20 | 0.90 | .76 |
| 45-54 | | 0.74 | −1.24 | 0.69 | .53 |
| 55-64 | | 0.96 | −0.99 | 0.95 | .93 |
| Gender, female | Male | 2.54 | 0.18 | 1.78 | .02 |
| Geographical region | | | | | |
| North Central | South | 0.50 | −1.42 | −0.03 | .05 |
| Northeast | | 0.27 | −2.78 | −0.22 | .04 |
| West | | 0.75 | −0.89 | 0.29 | .34 |
| ECI score | | | | | |
| 1 | ECI = 0 | 0.91 | −0.79 | 0.60 | .80 |
| 2 | | 0.77 | −1.17 | 0.63 | .57 |
| ≥3 | | 0.41 | −2.06 | 0.23 | .13 |
| Index year | | | | | |
| 2013 | 2016 | 0.91 | −1.73 | 1.30 | .90 |
| 2014 | | 0.80 | −0.89 | 0.46 | .51 |
| 2015 | | 0.80 | −0.91 | 0.48 | .53 |
| Comorbid conditions | | | | | |
| Diabetes | Yes vs no | 0.98 | −0.79 | 0.77 | .95 |
| CVD | | 1.08 | −0.65 | 0.84 | .83 |
| Hypertension | | 1.11 | −0.57 | 0.77 | .77 |
| Cancer | | 0.69 | −1.44 | 0.87 | .53 |
| Asthma | | 1.64 | −0.55 | 1.53 | .35 |
| COPD | | 0.47 | −1.60 | 0.12 | .08 |
| Anemia | | 0.77 | −1.16 | 0.71 | .57 |
| Rheumatoid vasculitis | | 0.86 | −1.76 | 1.88 | .87 |
| Osteoporosis | | 0.70 | −0.89 | 0.19 | .20 |
| Depression | | 1.47 | −0.47 | 1.25 | .38 |
| Mental illness | | 0.78 | −0.89 | 0.42 | .46 |

CVD indicates cardiovascular disease;
COPD, chronic obstructive pulmonary disorder;
ECI, Elixhauser comorbidity index

Among Optum patients, females (OR: 2.54; P=0.02) had increased odds of dose escalation compared with males, while patients from north-central (OR: 0.50; P=0.05) and north-eastern (OR: 0.27; P=0.04) regions had lower odds of dose escalation than patients from the south (Table 4). Other factors were not significant.

When the Cox model was utilized among patients with dose escalation, Truven patients from the northeast had an increased hazard ratio (HR) of dose escalation than patients from the south (HR: 1.82; P=0.01). Optum patients with depression had an increased HR of dose escalation compared with patients with no depression (HR: 3.51; P=0.04), and patients with an index year of 2014 or 2015 had a lower HR of dose escalation compared with patients with an index year of 2016 (HR: 0.33; P=0.01 and HR: 0.35, respectively; P=0.01) (Table 5).

TABLE 5
Cox Regression for Time to (First) Dose Escalation
Among Truven and Optum Patients Who Escalated Dose

| | | Hazard | 95% hazard ratio | | P |
|---|---|---|---|---|---|
| Parameter | Reference | ratio | confidence limits | | value |
| Truven MarketScan | | | | | |
| Index combination | Yes vs no | 0.96 | −0.49 | 0.41 | .85 |
| therapy | | | | | |
| Corticosteroid use | | 1.03 | −0.29 | 0.34 | .87 |
| Commercial | Medicare | 1.54 | −0.44 | 1.31 | .33 |
| Age, years | | | | | |
| 35-44 | 18-34 years | 1.10 | −0.50 | 0.70 | .75 |
| 45-54 | | 1.05 | −0.51 | 0.61 | .86 |
| 55-64 | | 1.48 | −0.15 | 0.93 | .15 |
| Gender, female | Male | 1.05 | −0.34 | 0.44 | .80 |
| Geographical region | | | | | |
| North central | South | 1.18 | −0.24 | 0.57 | .42 |
| Northeast | | 1.82 | 0.16 | 1.04 | .01 |
| West | | 0.80 | −0.62 | 0.18 | .28 |
| Unknown | | 0.40 | −2.04 | 0.21 | .11 |
| ECI score | | | | | |
| 1 | ECI = 0 | 1.28 | −0.17 | 0.66 | .24 |
| 2 | | 1.06 | −0.52 | 0.64 | .84 |
| ≥3 | | 0.91 | −0.83 | 0.65 | .81 |
| Index year | | | | | |
| 2013 | 2016 | 0.77 | −0.88 | 0.36 | .41 |
| 2014 | | 0.87 | −0.59 | 0.30 | .53 |
| 2015 | | 0.99 | −0.47 | 0.45 | .97 |
| Comorbid conditions | | | | | |
| Diabetes | Yes vs no | 1.10 | −0.55 | 0.75 | .77 |
| CVD | | 1.60 | −0.01 | 0.95 | .06 |
| Hypertension | | 1.01 | −0.40 | 0.43 | .95 |
| Cancer | | 1.09 | −0.97 | 1.14 | .88 |
| Asthma | | 2.34 | −0.06 | 1.76 | .07 |
| COPD | | 0.53 | −1.35 | 0.08 | .08 |
| Anemia | | 0.96 | −0.66 | 0.58 | .90 |
| Rheumatoid vasculitis | | 2.31 | −0.53 | 2.20 | .23 |
| Osteoporosis | | 0.90 | −0.49 | 0.27 | .58 |
| Depression | | 1.04 | −0.54 | 0.62 | .89 |
| Mental illness | | 1.02 | −0.43 | 0.47 | .93 |
| Optum | | | | | |
| Index combination | Yes vs no | 1.08 | −0.52 | 0.67 | .81 |
| therapy | | | | | |
| Corticosteroid use | | 1.26 | −0.33 | 0.78 | .42 |
| Commercial | Medicare | 1.38 | −0.65 | 1.29 | .52 |
| Age, years | | | | | |
| 35-44 | 18-34 years | 0.69 | −1.36 | 0.61 | .45 |
| 45-54 | | 0.66 | −1.36 | 0.54 | .39 |
| 55-64 | | 0.90 | −1.06 | 0.84 | .82 |
| Gender, female | Male | 1.34 | −0.60 | 1.19 | .52 |
| Geographical region | | | | | |
| North central | South | 1.54 | −0.35 | 1.21 | .28 |
| Northeast | | 2.02 | −0.76 | 2.16 | .35 |
| West | | 1.07 | −0.56 | 0.68 | .84 |
| ECI score | | | | | |
| 1 | ECI = 0 | 1.72 | −0.21 | 1.30 | .16 |
| 2 | | 0.88 | −1.08 | 0.82 | .79 |
| ≥3 | | 2.71 | −0.34 | 2.34 | .15 |
| Index year | | | | | |
| 2013 | 2016 | 0.55 | −2.16 | 0.96 | .45 |
| 2014 | | 0.33 | −1.87 | −0.32 | .01 |
| 2015 | | 0.35 | −1.86 | −0.24 | .01 |
| Comorbid conditions | | | | | |
| Diabetes | Yes vs no | 1.08 | −0.83 | 0.99 | .86 |
| CVD | | 0.69 | −1.31 | 0.58 | .45 |
| Hypertension | | 1.63 | −0.25 | 1.23 | .20 |
| Cancer | | 1.28 | −1.05 | 1.54 | .71 |
| Asthma | | 0.92 | −1.25 | 1.09 | .89 |
| COPD | | 1.35 | −0.61 | 1.22 | .52 |
| Anemia | | 2.45 | −0.20 | 1.99 | .11 |
| Rheumatoid vasculitis | | 1.40 | −1.45 | 2.12 | .71 |
| Osteoporosis | | 0.75 | −0.88 | 0.30 | .34 |
| Depression | | 3.51 | 0.09 | 2.42 | .04 |
| Mental illness | | 0.61 | −1.18 | 0.20 | .17 |

CVD indicates cardiovascular disease; COPD, chronic obstructive pulmonary disorder; ECI, Elixhauser comorbidity index.

Real-World Dose Modification Patterns of Subcutaneous Tocilizumab Among Patients with Rheumatoid Arthritis

TCZ has been approved in multiple countries for adults with moderate-to-severe RA (among other indications), who have had an inadequate response to >1 DMARD. Over the past decade, multiple studies have demonstrated the safety and effectiveness of TCZ in patients with RA.7-12 Dose modification patterns among patients with RA receiving IV TCZ have been examined; however, similar data among patients with RA receiving SC TCZ is limited. Present study is among the first to investigate SC TCZ dose modification in a real-world setting, and found that many patients utilized a QW dose of SC TCZ either at initiation or upon escalation, while few patients who started at the QW dose of SC TCZ had dose reduction.

The dose escalation patterns observed in this study are aligned with a study from Pappas and colleagues that prospectively looked at dosing patterns of IV TCZ in patients with RA from the US, and found that 51.6% of patients escalated from 4 mg/kg to 8 mg/kg. Although there was a difference in the mode of TCZ administration between the current study and that by Pappas and colleagues, both studies demonstrated that around 50% of patients on TCZ (IV or SC) require escalation to the higher dose.

Other studies have examined clinical outcomes in patients receiving the lower dose of IV TCZ versus the higher dose of IV TCZ. A double-blind, randomized, controlled clinical trial found that patients receiving a higher dose of IV TCZ (8 mg/kg) achieved a greater reduction in Disease Activity Score-28 (DAS-28) than patients receiving IV TCZ 2 mg/kg or 4 mg/kg. In addition, two randomized, double-blind, placebo-controlled trials demonstrated that more patients achieved American College of Rheumatology responses at 6 months with an IV TCZ 8 mg/kg dose than patients receiving an IV TCZ 4 mg/kg dose.

An open-label extension study among Japanese patients with RA, has examined the efficacy of SC TCZ QW versus SC TCZ Q2W, and found that patients who received SC TCZ QW had a greater improvement in DAS-28 scores than those who received SC TCZ Q2W. Although our study did not examine physician reasoning for dose escalation, the studies discussed here may provide some insights into why physicians escalated the dose of SC TCZ in almost half of the patients.

The other half of the patients in this study were started on the higher dose of SC TCZ 162 mg QW. However, only a small proportion of these patients (Truven, 3%; and Optum, 4%) had a reduction in SC TCZ dose. Of the patients who escalated their dose, the time to dose escalation was observed at around 4 months in both Truven and Optum patients. These results correlate with US treatment guidelines, which recommend that a therapeutic treatment should be given for at least 3 months before therapy escalation is considered. Among Truven patients, corticosteroid use within 90 days of the index date, aged 35-44 years, and presence of anemia had an OR of <1.0 for dose escalation. Female patients in both Optum and Truven had an increased OR of dose escalation; this was significant in Optum patients.

Of note, when the Cox model was utilized among patients with dose escalation, Optum patients with depression had an increased risk of dose escalation. In patients with RA, depression is a common disorder, affecting between 14% and 39% of patients. The co-occurrence of RA and depression has been found to be associated with increased levels of pain, fatigue, and disease activity, which may lead the physician to increase a patient's dose in order to control disease symptoms and improve quality of life, and could explain the results observed in the current study. In addition, Optum patients who initiated SC TCZ therapy in 2014 or 2015 had a lower risk of dose escalation than patients who initiated SC TCZ therapy in 2016. The greater number of Optum patients initiating SC TCZ in 2014 and 2015 following the approval of SC TCZ in 2013 would explain why there was a lower risk of dose escalation in these years compared with 2016. Meanwhile, the lower uptake of SC TCZ in 2016 among Optum patients could be due to increased experience of rheumatologists in relation to dose escalation among patients with RA receiving a lower dose of SC TCZ.

These study findings must be interpreted in light of the limitations. Firstly, retrospective observational studies are subject to uncertainty due to the generalizability of findings. The study sample was drawn from a population of commercially insured patients in the US and may not be generalizable to all patients with RA, nor other countries. In addition, the small sample size means that the study results should be interpreted with caution. Finally, the study only examined the administrative pharmacy claims for patients who initiated SC TCZ. Therefore, it was not possible to determine the exact reasoning behind the trends observed with regard to escalation and reduction in dosing.

### Conclusions

Using real-world data, this study demonstrated that overall, the utilization of a QW dose of SC TCZ either at initiation or upon escalation was 60% and 68% in Truven and Optum patients, respectively. Dose escalation of SC TCZ occurred in more than one-third of patients who initiated a Q2W dose of SC TCZ, and time to dose escalation was approximately 4 months. By contrast, <5% of patients starting at the QW dose had dose reduction of SC TCZ to Q2W. These results indicate that physicians appear to take advantage of the option to increase SC TCZ dose based on clinical response, but few choose to reduce the dose of SC TCZ, resulting in many patients on SC TCZ ultimately receiving the higher dose.

### REFERENCES

1. Singh J A, Saag K G, Bridges S L, Jr., et al. 2015 American College of Rheumatology guideline for the treatment of rheumatoid arthritis. Arthritis Rheumatol 2016;68:1-26. 10.1002/art.39480
2. Smolen J S, Landewé R, Bijlsma J, et al. EULAR recommendations for the management of rheumatoid arthritis with synthetic and biological disease-modifying antirheumatic drugs: 2016 update. Ann Rheum Dis 2017; 76:960-977. 10.1136/annrheumdis-2016-210715
3. Nishimoto N, Kishimoto T. Humanized antihuman IL-6 receptor antibody, tocilizumab. Handb Exp Pharmacol 2008:151-160. 10.1007/978-3-540-73259-4_7
4. Actemra Prescribing Information Genentech. Actemra (tocilizumab): full prescribing information. 2017. https://www.accessdatafda.gov/drugsatfda_docs/label/2017/125276s1141bl.pdf. Accessed Sep. 17, 2018.
5. United States Census Bureau. Census Regions and Divisions of the United States. 2018. https://www2.census.gov/geo/pdfs/maps-data/maps/reference/us_regdiv.pdf. Accessed Apr. 8, 2019.
6. Elixhauser A, Steiner C, Harris D R, Coffey R M. Comorbidity measures for use with administrative data. Med Care 1998; 36:8-27.
7. Jones G, Sebba A, Gu J, et al. Comparison of tocilizumab monotherapy versus methotrexate monotherapy in patients with moderate to severe rheumatoid arthritis: the AMBITION study. Ann Rheum Dis 2010; 69:88-96. 10.1136/ard.2008.105197
8. Genovese M C, McKay J D, Nasonov E L, et al. Interleukin-6 receptor inhibition with tocilizumab reduces disease activity in rheumatoid arthritis with inadequate response to disease-modifying antirheumatic drugs: the tocilizumab in combination with traditional disease-modifying antirheumatic drug therapy study. Arthritis Rheum 2008; 58:2968-2980. 10.1002/art.23940
9. Smolen J S, Beaulieu A, Rubbert-Roth A, et al. Effect of interleukin-6 receptor inhibition with tocilizumab in patients with rheumatoid arthritis (OPTION study): a double-blind, placebo-controlled, randomised trial. Lancet 2008; 371:987-997. 10.1016/s0140-6736(08)60453-5
10. Kremer J M, Blanco R, Brzosko M, et al. Tocilizumab inhibits structural joint damage in rheumatoid arthritis patients with inadequate responses to methotrexate: results from the double-blind treatment phase of a randomized placebo-controlled trial of tocilizumab safety and prevention of structural joint damage at one year. Arthritis Rheum 2011; 63:609-621. 10.1002/art.30158
11. Emery P, Keystone E, Tony H P, et al. IL-6 receptor inhibition with tocilizumab improves treatment outcomes in patients with rheumatoid arthritis refractory to anti-tumour necrosis factor biologicals: results from a 24-week multicentre randomised placebo-controlled trial. Ann Rheum Dis 2008; 67:1516-1523. 10.1136/ard.2008.092932
12. Kaneko A. Tocilizumab in rheumatoid arthritis: efficacy, safety and its place in therapy. Ther Adv Chronic Dis 2013; 4:15-21. 10.1177/2040622312466908
13. Dhillon S. Intravenous tocilizumab: a review of its use in adults with rheumatoid arthritis. BioDrugs 2014; 28:75-106. 10.1007/s40259-013-0076-8
14. Pappas D A, John A, Curtis J R, et al. Dosing of intravenous tocilizumab in a real-world setting of rheumatoid arthritis: analyses from the Corrona Registry. Rheumatol Ther 2016; 3:103-115. 10.1007/s40744-016-0028-0
15. Maini R N, Taylor P C, Szechinski J, et al. Double-blind randomized controlled clinical trial of the interleukin-6 receptor antagonist, tocilizumab, in European patients with rheumatoid arthritis who had an incomplete response to methotrexate. Arthritis Rheum 2006; 54:2817-2829. 10.1002/art.22033
16. Ogata A, Tanaka Y, Ishii T, et al. Long-term safety and efficacy of weekly subcutaneous tocilizumab monotherapy in patients with rheumatoid arthritis who had an inadequate response to subcutaneous tocilizumab every other week: Results from the open-label extension of the SHINOBI study. Mod Rheumatol 2018:1-8. 10.1080/14397595.2018.1533514
17. van Herwaarden N, Herfkens-Hol S, van der Maas A, et al. Dose reduction of tocilizumab in rheumatoid arthritis patients with low disease activity. Clin Exp Rheumatol 2014; 32:390-394.
18. Nadkarni A, McMorrow D, Patel C, Fowler R, Smith D. Incidence of dose escalation and impact on biologic costs among patients with rheumatoid arthritis treated with three intravenous agents. J Comp Eff Res 2017; 6:671-682. 10.2217/cer-2016-0090
19. Garces K, Thorne C, Boire G, et al. Comparison of dose escalation and costs of dose escalation between patients with rheumatoid arthritis initiating biologic treatment with etanercept, adalimumab, or infliximab. Value Health 2016; 19:A229-A230. 10.1016/j.jval.2016.03.1129
20. van Vollenhoven R F. Sex differences in rheumatoid arthritis: more than meets the eye. BMC Med 2009; 7:12-12. 10.1186/1741-7015-7-12
21. Matcham F, Norton S, Scott D L, Steer S, Hotopf M. Symptoms of depression and anxiety predict treatment response and long-term physical health outcomes in rheumatoid arthritis: secondary analysis of a randomized controlled trial. Rheumatology (Oxford) 2016; 55:268-278. 10.1093/rheumatology/kev306
22. Matcham F, Rayner L, Steer S, Hotopf M. The prevalence of depression in rheumatoid arthritis: a systematic review and meta-analysis. Rheumatology (Oxford) 2013; 52:2136-2148. 10.1093/rheumatology/ket169
23. Kojima M, Kojima T, Suzuki 5, et al. Depression, inflammation, and pain in patients with rheumatoid arthritis. Arthritis Rheum 2009; 61:1018-1024. 10.1002/art.24647
24. Matcham F, Ali S, Hotopf M, Chalder T. Psychological correlates of fatigue in rheumatoid arthritis: a systematic review. Clin Psychol Rev 2015; 39:16-29. 10.1016/j.cpr.2015.03.004
25. Overman C L, Bossema E R, van Middendorp H, et al. The prospective association between psychological distress and disease activity in rheumatoid arthritis: a multilevel regression analysis. Ann Rheum Dis 2012; 71:192-197. 10.1136/annrheumdis-2011-200338
26. Drugs.com. FDA approves new subcutaneous formulation of actemra. 2013. https://www.drugs.com/newdrugs/genentech-gains-fda-approval-new-subcutaneous-formulation-actemra-adult-patients-living-moderately-3947.html. Accessed Jan. 4, 2019.

1. An antibody for use in treatment of rheumatoid arthritis in a subject in need thereof, wherein,
(i) the subject has not previously been administered tocilizumab or has been administered tocilizumab for less than three months, and does not have anemia; and
(ii) the antibody is administered at
a. 162 mg of tocilizumab once per week subcutaneously to the subject; or
b. 8 mg/kg of tocilizumab once every 4 weeks intravenously to the subject.

2. An antibody for use in treatment of rheumatoid arthritis in a subject in need thereof, wherein
(i) the subject has not previously been administered tocilizumab or has been administered tocilizumab for less than three months, and is from 18 to 34 years old; and
(ii) the antibody is administered at
a. 162 mg of tocilizumab once per week subcutaneously to the subject; or
b. 8 mg/kg of tocilizumab once every 4 weeks intravenously to the subject.

3. An antibody for use in treatment of rheumatoid arthritis in a subject in need thereof, wherein
(i) the subject has not previously been administered tocilizumab or has been administered tocilizumab for less than three months, and has not been administered a corticosteroid within 90 days; and
(ii) the antibody is administered at
a. 162 mg of tocilizumab once per week subcutaneously to the subject; or
b. 8 mg/kg of tocilizumab once every 4 weeks intravenously to the subject.

4. An antibody for use in treatment of rheumatoid arthritis in a subject in need thereof, wherein
(i) the subject has not previously been administered tocilizumab or has been administered tocilizumab for less than three months, and has depression; and
(ii) the antibody is administered at
a. 162 mg of tocilizumab once per week subcutaneously to the subject; or
b. 8 mg/kg of tocilizumab once every 4 weeks intravenously to the subject.

5. The antibody for use according to any one of claims 1-4, wherein the antibody is administered at 162 mg of tocilizumab once per week subcutaneously to the subject.

6. The antibody for use according to any one of claims 1-5, wherein the antibody is administered at 8 mg/kg of tocilizumab once every 4 weeks intravenously to the subject.

7. The antibody for use according to any one of claims 1-6, wherein the subject has moderately-to-severely active rheumatoid arthritis.

8. The antibody for use according to any one of claims 1-7, wherein the subject has not been administered sarilumab.

9. The antibody for use according to any one of claims 1-8, wherein the subject weighs less than 100 kg.

10. The antibody for use according to any one of claims 1-9, wherein the subject does not have ankylosing spondylitis, Crohn's disease, juvenile idiopathic arthritis, psoriasis, psoriatic arthritis, ulcerative colitis, chronic lymphocytic leukemia, non-Hodgkin's lymphoma, or giant-cell arteritis.

11. The antibody for use according to any one of claims 1, 2, and 5-10, wherein the subject does not have anemia and is from 18 to 34 years old.

12. The antibody for use according to any one of claims 1, 3, and 5-10, wherein the subject does not have anemia and has not been administered a corticosteroid within 90 days.

13. The antibody for use according to any one of claims 1, 2, and 5-10, wherein the subject is from 18 to 34 years old and has not been administered a corticosteroid within 90 days.

14. The antibody for use according to any one of claims 1-3 and 5-10, wherein the subject does not have anemia, has not been administered a corticosteroid within 90 days, and is from 18 to 34 years old.

15. The antibody for use according to any one of claims 1 and 4-10, wherein the subject does not have anemia and has depression.

16. The antibody for use according to any one of claims 3-10, wherein the subject has depression and has not been administered a corticosteroid within 90 days.

17. The antibody for use according to any one of claims 2 and 4-10, wherein the subject is from 18 to 34 years old and has depression.

18. The antibody for use according to any one of claims 1, 2, and 4-10, wherein the subject does not have anemia, has depression and is from 18 to 34 years old.

19. The antibody for use according to any one of claims 1 and 4-10, wherein the subject does not have anemia, has depression, and has not been administered a corticosteroid within 90 days.

20. The antibody for use according to any one of claims 1, 2, and 4-10, wherein the subject is from 18 to 34 years old, has depression, and has not been administered a corticosteroid within 90 days.

21. The antibody for use according to any one of claims 1-20, wherein the subject subject does not have anemia, has not been administered a corticosteroid within 90 days, is from 18 to 34 years old, and has depression.

22. The antibody for use according to any one of claims 3, 5-10, 12, 13, 14, 16, and 19-20, wherein the within 90 days is within 90 days of the subject's first administration of tocilizumab.

23. The antibody for use according to any one of claims 3, 5-10, 12, 13, 14, 16, and 19-22, wherein the corticosteroid is prednisone.

24. The antibody for use according to any one of claims 1-23, wherein the subject is not administered any other DMARD in course of administration with tocilizumab.

25. The antibody ritis according to any one of claims 1-23, wherein the subject is also administered one or more additional DMARDs with tocilizumab.

26. The antibody for use according to claim 25, wherein the one or more additional DMARDs comprise methotrexate.

27. The antibody for use according to any one of claims 1-26, wherein the subject previously had an inadequate response to a conventional synthetic DMARD or a biologic DMARD.

28. The antibody for use according to claim 27, wherein the conventional synthetic DMARD is methotrexate.

29. The antibody for use according to claim 27, wherein the biologic DMARD is a TNFα inhibitor.

30. The antibody for use according to claim 29, where the TNFα inhibitor is adalimumab.

31. The antibody for use according to any one of claims 1-30, wherein the subject has not previously been administered tocilizumab.

32. The antibody for use according to any one of claims 1-30, wherein the subject has been administered tocilizumab for less than three months.

33. The antibody for use according to claim 32, wherein the subject has been administered tocilizumab for less than two months.

34. The antibody for use according to claim 32, wherein the subject has been administered tocilizumab for less than one month.

35. The antibody for use according to any one of claims 1-35, wherein the subject is female.

36. A method of treating rheumatoid arthritis, comprising
(iii) selecting a subject who has not previously been administered tocilizumab or who has been administered tocilizumab for less than three months, and who does not have anemia; and
(iv) administering
c. 162 mg of tocilizumab once per week to the subject, wherein the tocilizumab is administered subcutaneously; or
d. 8 mg/kg of tocilizumab once every 4 weeks to the subject, wherein the tocilizumab is administered intravenously.

37. A method of treating rheumatoid arthritis, comprising
(iii) selecting a subject who has not previously been administered tocilizumab or who has been administered tocilizumab for less than three months, and who is from 18 to 34 years old; and
(iv) administering
c. 162 mg of tocilizumab once per week to the subject, wherein the tocilizumab is administered subcutaneously; or
d. 8 mg/kg of tocilizumab once every 4 weeks to the subject, wherein the tocilizumab is administered intravenously.

38. A method of treating rheumatoid arthritis, comprising
(iii) selecting a subject who has not previously been administered tocilizumab or who has been administered tocilizumab for less than three months, and who has not been administered a corticosteroid within 90 days; and
(iv) administering
c. 162 mg of tocilizumab once per week to the subject, wherein the tocilizumab is administered subcutaneously; or
d. 8 mg/kg of tocilizumab once every 4 weeks to the subject, wherein the tocilizumab is administered intravenously.

39. A method of treating rheumatoid arthritis, comprising
(iii) selecting a subject who has not previously been administered tocilizumab or who has been administered tocilizumab for less than three months, and who has depression; and
(iv) administering
c. 162 mg of tocilizumab once per week to the subject, wherein the tocilizumab is administered subcutaneously; or
d. 8 mg/kg of tocilizumab once every 4 weeks to the subject, wherein the tocilizumab is administered intravenously.

40. The method of any one of claims 36-39, wherein step (ii) comprises administering 162 mg of tocilizumab once per week to the subject, wherein the tocilizumab is administered subcutaneously.

41. The method of any one of claims 36-40, wherein step (ii) comprises administering 8 mg/kg of tocilizumab once every 4 weeks to the subject, wherein the tocilizumab is administered intravenously.

42. The method of any one of claims 36-41, wherein the subject has moderately-to-severely active rheumatoid arthritis.

43. The method of any one of claims 36-42, wherein the subject has not been administered sarilumab.

44. The method of any one of claims 36-43, wherein the subject weighs less than 100 kg.

45. The method of any one of claims 36-44, wherein the subject does not have ankylosing spondylitis, Crohn's disease, juvenile idiopathic arthritis, psoriasis, psoriatic arthritis, ulcerative colitis, chronic lymphocytic leukemia, non-Hodgkin's lymphoma, or giant-cell arteritis.

46. The method of any one of claims 36, 37, and 40-45, wherein the subject is selected if the subject does not have anemia and is from 18 to 34 years old.

47. The method of any one of claims 36, 38, and 40-45, wherein the subject is selected if the subject does not have anemia and has not been administered a corticosteroid within 90 days.

48. The method of any one of claims 36, 37, and 40-45, wherein the subject is selected if the subject is from 18 to 34 years old and has not been administered a corticosteroid within 90 days.

49. The method of any one of claims 36, 37 and 39-45, wherein the subject is selected if the subject does not have anemia, has not been administered a corticosteroid within 90 days, and is from 18 to 34 years old.

50. The method of any one of claims 36 and 39-45, wherein the subject is selected if the subject does not have anemia and has depression.

51. The method of any one of claims 38-45, wherein the subject is selected if the subject has depression and has not been administered a corticosteroid within 90 days.

52. The method of any one of claims 37 and 39-45, wherein the subject is selected if the subject is from 18 to 34 years old and has depression.

53. The method of any one of claims 36, 37, and 39-45, wherein the subject is selected if the subject does not have anemia, has depression and is from 18 to 34 years old.

54. The method of any one of claims 36 and 39-45, wherein the subject is selected if the subject does not have anemia, has depression, and has not been administered a corticosteroid within 90 days.

55. The method of any one of claims 36, 37, and 39-45, wherein the subject is selected if the subject is from 18 to 34 years old, has depression, and has not been administered a corticosteroid within 90 days.

56. The method of any one of claims 36-55, wherein the subject is selected if the subject does not have anemia, has not been administered a corticosteroid within 90 days, is from 18 to 34 years old, and has depression.

57. The method of any one of claims 38, 40-45, 47, 48, 49, 51, and 54-55, wherein the within 90 days is within 90 days of the subject's first administration of tocilizumab.

58. The method of any one of claims 38, 40-45, 47, 48, 49, 51, and 54-55, wherein the within 90 days is within 90 days of the selection.

59. The method of any one of claims 38, 40-45, 47, 48, 49, 51, and 54-55, wherein the corticosteroid is prednisone.

60. The method of any one of claims 36-59, wherein the subject is not administered any other DMARD in course of administration with tocilizumab.

61. The method of any one of claims 36-59, wherein the subject is also administered one or more additional DMARDs with tocilizumab.

62. The method of claim 61, wherein the one or more additional DMARDs comprise methotrexate.

63. The method of any one of claims 36-62, wherein the subject previously had an inadequate response to a conventional synthetic DMARD or a biologic DMARD.

64. The method of claim 63, wherein the conventional synthetic DMARD is methotrexate.

65. The method of claim 63, wherein the biologic DMARD is a TNFα inhibitor.

66. The method of claim 65, where the TNFα inhibitor is adalimumab.

67. The method of any one of claims 36-66, wherein the subject has not previously been administered tocilizumab.

68. The method of any one of claims 36-66, wherein the subject has been administered tocilizumab for less than three months.

69. The method of claim 66, wherein the subject has been administered tocilizumab for less than two months.

70. The method of claim 66, wherein the subject has been administered tocilizumab for less than one month.

71. The method of any one of claims 36-70, wherein the subject is female.
